(19) United States
(12) Patent Application Publication
(10) Pub. No.: US 2007/0082050 A1
(43) Pub. Date: Apr. 12, 2007

(54) Modified release formulations of antihypertensive drugs

(76) Inventors: S. Rao Cherukuri, Vienna, VA (US); Vittorino Ravelli, Milano (IT)

Correspondence Address: THORPE NORTH & WESTERN, LLP., 8180 SOUTH 700 EAST, SUITE 200, SANDY, UT, 84070, US

(21) Appl. No.: 11/443,671

(22) Filed: May 30, 2006

Related U.S. Application Data
(60) Provisional application No. 60/685,788, filed on May 31, 2005.

(51) Int. Cl. A61K 9/22

(52) U.S. Cl. 424/468

(57) ABSTRACT

Modified or extended release formulations containing propanolol compounds and associated methods are disclosed and described. In some aspects, such formulations may be substantially bioequivalent to known FDA approved propanolol formulations such as INDERAL LA®.

### PRIORITY DATA

This application claims the benefit of U.S. Provisional Patent Application Ser. No. 60/685,788, filed on May 31, 2005, which is incorporated herein by reference.

### FIELD OF THE INVENTION

The present invention relates to propranolol compound containing formulations with desired in-vitro and in-vivo characteristics which are simple to formulate and economical to manufacture on a commercial scale. Accordingly, the present invention involves the field of pharmaceutical sciences.

### BACKGROUND OF THE INVENTION

Modified release propranolol formulations are desirable because they can achieve better control of hypertension for a longer period of time compared to immediate release formulations which often require multiple dosings in a single day. Examples of various known modified release propranolol formulations may be found in U.S. Pat. Nos. 6,337,091; 5,968,554; and 5,508,043, each of which are incorporated by reference.

While propanolol has been used for many years as an active agent in an immediate release dosage form to control hypertension, it has proven very difficult to put into a modified release formulation. So much so, that at present only one modified release propanolol has ever been successfully approved by the FDA and marketed to the public. This product is marketed by Wyeth under the tradename INDERAL LA®.

No generic drug company has ever successfully created a modified release propanolol formulation that has been approved by the FDA as bioequivalent to INDERAL LA®. This could be due to the relative high water solubility of the propranolol hydrochloride molecule which makes it difficult to control its release. This could also be due to the extreme first-pass effect (the extensive liver metabolism) propranolol experiences in-vivo. Perhaps another factor is the complexity of these prior art disclosures in terms of their formulation and manufacturing steps.

Accordingly, there is an undisputed commercial need for a modified propranolol dosage form that is pharmaceutically equivalent to the FDA-approved brand product INDERAL LA®.

### SUMMARY OF THE INVENTION

Methods are provided for formulating and manufacturing modified release propranolol dosage forms for oral delivery. Also provided herein are dosage forms thus produced. Methods are also provided for administering such modified dosage forms to a mammal such as humans and members of the animal kingdom. In some aspects, the dosage form is a capsule. In some aspects, the dosage form is a tablet. The amount of propranolol hydrochloride per dosage form can be, as stated conventionally, from about 60 mg to about 300 mg, including specific intermediate amounts such as 80 mg, 120 mg, 160 mg, 200 mg and 240 mg.

Alternatively, these dosage forms provide a dissolution profile such that: about 10-30% of the drug is released by 90 minutes; about 30% to about 60% of the drug is released by 4 hrs; and about 50% to about 80% of the drug is released by 8 hrs; when dissolution test is performed using pH 6.8 phosphate buffer and simulated intestinal fluid without pancreatin.

In yet another aspect, these dosage forms provide a dissolution profile such that: about 20% to about 45% of the drug is released by 90 minutes; about 40% to about 75% of the drug is released by 4 hrs; about 60% to about 90% of the drug is released by 8 hrs, when dissolution test is performed using pH 1.2 simulated gastric fluid without pepsin.

In one other aspect, these dosage forms provide a dissolution profile such that: about 20 to about 50% of the drug is released by 90 minutes; about 60% to about 90% of the drug is released by 4 hrs; about 70% to about 100% of the drug is released by 8 hrs when dissolution test is performed using pH 4.5 phosphate buffer.

These dosage forms provide a dissolution profile such that about 10-25% of the drug is released by 90 minutes; about 25-55% of the drug is released by 4 hrs; about 40-70% of the drug is released by 6 hrs; and about 60% to about 80% of the drug is released by 8 hrs, under USP dissolution conditions with a pH of 1.2 for 2 hours followed by a pH of 6.8 for the rest of the time, using Type 1 dissolution apparatus being operated at about 37° C., using a volume of about 900 ml of the dissolution medium being stirred at a speed of 100 rpm.

In some aspects, the dosage form provides peak blood plasma concentrations at about 6 hrs after administration to a mammal. The dosage forms may be used to treat hypertension, angina, migraine, hypertrophic subaortic stenosis, among others.

In one aspect, the method comprises the following steps:
a) preparing a mixture of propranolol hydrochloride and one or more pharmaceutically acceptable excipients to form a propranolol-excipient mixture;
b) granulate the propranolol-excipient mixture in the presence of a water-insoluble polymer to produce propranolol granulates;
c) spheronize and extrude the propranolol granulates to produce propranolol cores, and optionally drying and sieving said cores;
d) prepare a dispersion of a water-insoluble polymer and a water-swellable polymer to produce a coating polymer dispersion;
e) coat said propranolol cores with said coating polymer dispersion to obtain coated propranolol cores; and
f) provide modified release propranolol capsules by filling empty capsules with coated propranolol cores.

In one aspect, the one or more pharmaceutically acceptable exicipients may be selected from the group consisting of: microcrystalline cellulose, dibasic calcium phosphate dihydrate, starch, sodium starch glycolate, crospovidone, croscarmellose sodium, magnesium stearate, lactose, maleic acid, colloidal silicon dioxide, talc, and glyceryl behenate, or a mixture thereof.

In another aspect, the water-insoluble polymer is selected from the group consisting of ethylcellulose, propylcellulose, isopropylcellulose, or a mixture thereof.

In another aspect, the water-swellable polymer is selected from the group consisting of methylcellulose (MC), carboxymethylcellulose (CMC), hydroxypropylcellulose (HPC), hydroxypropylmethylcellulose (HPMC), hydroxyethylcellulose (HEC); polyvinylpyrrolidone (PVP), polyvinyl alcohol (PVA); and acrylic acid polymer, methacrylic acid copolymers, ethyl acrylate-methyl methacrylate copolymers, or a mixture thereof.

In another aspect, the method comprises administering the dosage form prepared as above.

In one aspect the invention provides a dosage form of propranolol prepared according to the methods described herein.

In another aspect, the invention provides an article of manufacture comprising propranolol prepared in accordance with the methods described herein and accompanying labeling and packaging to enable the article of manufacture to be shipped interstate.

In another aspect, a modified release propranolol oral dosage form is provided comprising:
a) a therapeutically effective amount of propranolol hydrochloride, ranging from about 60 mg to about 300 mg per dosage unit, formulated into one or more cores comprising said propranolol and one or pharmaceutically acceptable excipients;
b) a release-modifying coat that substantially overlaps said core, wherein said coat comprises a mixture of a water-insoluble polymer and a water-swellable polymer; to provide a dissolution profile selected from the group consisting of:
   i) about 10-30% of the drug is released by 90 minutes; about 30% to about 60% of the drug is released by 4 hrs; and about 50% to about 80% of the drug is released by 8 hrs; when dissolution test is performed using pH 6.8 phosphate buffer and simulated intestinal fluid without pancreatin, using Type 1 dissolution apparatus being operated at about 37° C., using a volume of about 900 ml of the dissolution medium being stirred at a speed of 100 rpm;
   ii) about 20% to about 45% of the drug is released by 90 minutes; about 40% to about 75% of the drug is released by 4 hrs; about 60% to about 90% of the drug is released by 8 hrs, when dissolution test is performed using pH 1.2 simulated gastric fluid without pepsin, using Type 1 dissolution apparatus being operated at about 37° C., using a volume of about 900 ml of the dissolution medium being stirred at a speed of 100 rpm;
   iii) about 20 to about 50% of the drug is released by 90 minutes; about 60% to about 90% of the drug is released by 4 hrs; about 70% to about 100% of the drug is released by 8 hrs when dissolution test is performed using pH 4.5 phosphate buffer, using Type 1 dissolution apparatus being operated at about 37° C., using a volume of about 900 ml of the dissolution medium being stirred at a speed of 100 rpm; and
   iv) about 10-25% of the drug is released by 90 minutes; about 25-55% of the drug is released by 4 hrs; about 40-70% of the drug is released by 6 hrs; and about 60% to 5 about 80% of the drug is released by 8 hrs, under USP dissolution conditions with a pH of 1.2 for 2 hours followed by a pH of 6.8 for the rest of the time, using Type 1 dissolution apparatus being operated at about 37° C., using a volume of about 900 ml of the dissolution medium being stirred at a speed of 100 rpm,
wherein said dosage form further provides peak blood plasma concentrations at about 6 hrs after administration to a mammal.

The foregoing and other objects and aspects of the present invention are explained in detail in the detailed description and examples set forth below.

### BRIEF DESCRIPTION OF THE FIGURES

FIG. 1 is a graphical representation of dissolution testing results of a propanolol formulation prepared in accordance with one embodiment of the present invention. The data were obtained under pH 1.2, 4.5, and 6.8 conditions.

FIG. 2 is a graphical representation of dissolution testing results of a propanolol formulation prepared in accordance with another embodiment of the present invention. The data were obtained under USP conditions, where pH was maintained at 1.2 for two hours, followed by pH of 6.8 the rest of the time.

FIG. 3 is a graphical representation of dissolution testing results of a propanolol formulation prepared in accordance with one embodiment of the present invention using USP conditions, where pH was maintained at 1.2 for two hours, followed by pH of 6.8 the rest of the time.

### DETAILED DESCRIPTION OF THE INVENTION

Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a drug” includes reference to one or more of such drugs, and reference to “an excipient” includes reference to one or more of such excipients.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients.

As used herein, “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical, and medicinal arts.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, “propanolol” refers to a compound known by the IUPAC name of 1-(isopropylamino)-3(napthalen-1-yloxy) propan-2-ol and having the structure: 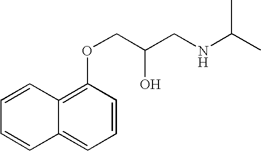
Propanolol has a CAS Registry number of 525-66-6. The term “propanolol” also refers to not only the above-recited compound, but also encompasses related compounds, such as analogs and homologs thereof, salts, such as acid addition salts thereof, prodrugs, isomers and metabolites thereof, as well as mixtures thereof as dictated by the context of its use. However, when referring to individual specific related compounds, or groups of compounds such as the acid addition salt propanolol hydrochloride, or Propanolol HCl, the specific technical name of each compound or molecule can also be used, or the group can be specifically named, such as “propanalol salts”. As such, inherent support for all such well known individual specific compound names in view of the above-recited definition, is considered to be included herein, though they may not each be expressly recited.

As used herein, “blood level” may be used interchangeably with terms such as blood plasma concentration, plasma level, plasma concentration, serum level, serum concentration, serum blood level and serum blood concentration.

As used herein, “oral dosage form” and the like refers to a formulation that is ready for administration to a subject through the oral route of administration. Examples of known oral dosage forms, include without limitation, tablets, capsules, caplets, powders, pellets, granules, etc. Such formulations also include multilayered tablets wherein a given layer may represent a different drug. In some aspects, powders, pellets, and granules may be coated with a suitable polymer or a conventional coating material to achieve, for example, greater stability in the gastrointestinal tract, or to achieve the desired rate of release. Moreover, capsules containing a powder, pellets or granules may be further coated. Tablets and caplets may be scored to facilitate division of dosing. Alternatively, the dosage forms of the present invention may be unit dosage forms wherein the dosage form is intended to deliver one therapeutic dose per administration.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier” and “carrier” may be used interchangeably, and refer to any inert and pharmaceutically acceptable material that has substantially no biological activity, and makes up a substantial part of the formulation.

The term “admixed” means that the drug and/or other ingredients can be dissolved, dispersed, or suspended in the carrier. In some cases, the drug may be uniformly admixed in the carrier.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

The term “modified release” as used herein refers to the drug release that is different from an immediate release. Typically, in an immediate release dosage form, about more than 80% of the drug is released from the dosage form in vitro within about 2 hrs. This release may be measured in terms of dissolution of the drug in the dissolution medium. In one aspect, the release is measured under USP conditions, i.e., where the pH is maintained at 1.2 for 2 hours, followed by a pH of 6.8 for the rest of the time. In another aspect, the release is measured at a pH of 1.2 for the entire period of measurement. Examples of such modified release include sustained release, slow-release, delayed-release, pulsatile release etc., which terms are generally known in the art and to the extent they mean a release other than an immediate release.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The Invention

The present invention provides modified release propranolol compound dosage forms with certain desirable in-vitro dissolution properties and in-vivo blood plasma concentrations.

In one aspect, the invention provides methods for formulating a modified release propranolol compound capsule dosage form. The capsule may contain one or more cores, depending on the dosage the capsule is intended to deliver, that comprise propranolol hydrochloride and one or more excipients. A variety of excipients commonly known in the pharmaceutical industry may be used. The cores are then coated with a mixture of polymers comprising at least a water-insoluble coating polymer and a water-swellable polymer. It has been discovered by the present inventors that this specific mixture of polymers provides the desired product with the desired in-vitro and in-vivo performance.

In one aspect, the cores may be prepared by the following process. Propranolol hydrochloride and one or more inert pharmaceutically acceptable excipients may be mixed thoroughly to achieve a substantially homogenous mixture. The excipients which may be employed are well known to those skilled in the art and include any conventional pharmaceutically acceptable tabletting excipients. Examples of suitable excipients include but are not limited to microcrystalline cellulose, dibasic calcium phosphate dihydrate, starch, sodium starch glycolate, crospovidone, croscarmellose sodium, magnesium stearate, lactose, maleic acid, colloidal silicon dioxide, talc, and glyceryl behenate, as well as mixtures and various combinations thereof

The mixing of the excipients and propranolol hydrochloride can be accomplished by using high shear granulators (mixers, blenders, etc). The homogenous mixture may be then processed into cores by a number of alternative processes such as granulation, spheronization, spheronization/extrusion, etc. These cores are then optionally dried. The drying process may provide certain advantages such as improvements in content uniformity, ease of handling, etc.

Alternatively, the propranolol hydrochloride and excipient mixture may be granulated with a water-insoluble polymeric dispersion to form granules of drug+excipient+water-insoluble polymer. The water-insoluble polymer may be in one aspect ethylcellulose. Ethylcellulose may be used at a concentration ranging from about 1-20% in a non-aqueous solvent such as ethanol, isopropanol, or a mixture thereof. In some aspects, the ethylcellulose concentration may have the following ranges: from about 1-10%; from about 5-15%; from about 5-10%; from about 3-8%; from about 4-7%. In another aspect, the ethylcellulose concentration is about 6%.

This drug+excipient+water-insoluble polymer granulate is then optionally dried to substantially remove any residual solvents. Then the granulates may be optionally wetted to facilitate spheronization to extrude granules into an extruder. The operating conditions of the spheronization and extrusion processes and equipment are generally well-known in the art. The spheronization process yields cores that may be optionally sieved to optimize desired core size.

The cores thus obtained by either of the above alternate processes are then coated with a mixture of polymers comprising a water-insoluble coating polymer and a water-swellable polymer. This coating substantially surrounds the core. Examples of water-insoluble polymers that can be used to make the coating include without limitation various water-insoluble celluloses, such as ethyl cellulose, propyl cellulose, etc. Examples of water-swellable polymers include without limitation, hydroxypropylmethylcellulose, gums, etc.

In one aspect, the coating mixture comprises HPMC and ethylcellulose dispersed in an aqueous or substantially non-aqueous solvent. A substantially non-aqueous solvent may be selected form a variety of solvents such as methanol, ethanol, isopropanol, acetone, or a mixture thereof. The HPMC and ethylcellulose may be selected from one of several grades that are commercially available, as described elsewhere in this application.

The amount of water-insoluble polymer in the coating may range from about 0.5% to about 10% of the modified release formulation. In some aspects, the amount of water-insoluble polymer in the coating may range as following: from about 1-10%; from about 2-8%; from about 2-6%; from about 1-5%; from about 1-3%; from about 2-3% of the modified release composition. In some specific aspects, the water-insoluble polymer in the coating may amount to about 2.5% of the modified release composition. These amounts are expressed as w/w %.

The amount of water-swellable polymer in the coating may range from about 0.1% to about 5% of the modified release formulation. In some aspects, the amount of water-swellable polymer in the coating may range as following: from about 0.5% to about 3%; from about 0.5% to about 2%; from about 0.5% to about 1.5% of the modified release composition. In some specific aspects, the water-swellable polymer in the coating may amount to about 1% of the modified release composition. These amounts are expressed as w/w %.

In one aspect, the ratio of water-insoluble polymer to the water-swellable polymer may be from about 80 to about 20. In another aspect, that ratio may be: from about 70 to about 30; from about 60 to about 40; from about 50 to about 50; from about 40 to about 60; from about 30 to 70; from about 20 to about 80.

The polymeric coating layer may be accomplished by directly applying the coating polymer mixture alone or together with a binder, either as a solution or as a powder. For example, the binder may be provided as a solution or as a dispersion and may be applied just prior to, or together with the polymer mixture. The polymer mixture may be applied as a dispersion (which may be a solution, suspension or as an emulsion) if the binder is provided as a solution or as a powder. Alternatively, the binder may be provided as a fine powder and the polymer mixture may be provided as a dispersion. Upon contact with the polymer dispersion, the binder powder may become a solution or suspension which then forms a binding film on the cores and thus facilitate the coating of the polymer onto the cores.

In some aspects, the particle size of the cores and/or coated cores ranges from about 750 uM to about 1200 uM. In some aspects, the particle size ranges from about 800 uM to about 1100 uM. In another aspect, the particle size ranges from about 900 uM to about 1100 uM. In one particular aspect, the particle size may range from about 1000 uM to about 1100 uM.

The polymeric coating layer may be applied to the core according to methods generally known in the art. For example, a two-step process, within which the steps may be repeated a sufficient number of times as necessary to build the thickness of the polymeric coating layer to achieve the desired in-vitro and in-vivo characteristics. In the first step, the core is wet with the binder dispersion which serves to adhere the powdered polymeric coating particles to the wet core. Suitable binder dispersions may include conventional pharmaceutically acceptable binder agents solubilized in a suitable solvent. Specific examples of binder agents include but are not limited to vinyl polymers, such as polyvinylpyrrolidone, polyvinyl alcohol, and the like; cellulosic polymers, such as HPMC, HEC, HPC, and the like; acrylic polymers and copolymers such as methacrylic acid copolymers, ethyl acrylate-methylmethacrylate copolymers, and the like; natural or synthetic gums, such as guar gum, arabic gum, xanthan gum, and the like; proteins or carbohydrates, such as gelatin, pectin, and the like; and mixtures thereof. In some aspects, polyvinylpyrrolidone is the preferred binder agent.

Suitable solvents for solubilizing the binder agents include solvents which are capable of substantially completely solubilizing the specific binder agent(s) selected and which are pharmaceutically and biologically acceptable for ingestion. Suitable solvents will be readily determinable by those skilled in the art. Water is currently the preferred solvent for solubilizing the binder agent. However, other examples of suitable solvents will be appreciated by those skilled in the art and are contemplated by the methods of the present invention.

The binder solution should be of sufficient viscosity to enable the wetting of the cores by any suitable wetting technique known to those skilled in the art. For example, the cores may be wetted with the binder solution by rotating the cores in a bath containing the binder solution. The cores may be suitably wetted by manual application of the binder dispersion by layer the binder solution over the cores as the cores are rotating in a conventional coating pan. Alternatively, the cores may be wetted by spraying the binder dispersion on the cores. In one aspect, the wetting step is advantageously carried out using conventional automated pan coating equipment wherein the cores are sprayed with the binder dispersion while rotating in the pan.

To provide the coating layer, the wetted cores may be coated with dry, powdered polymeric coating particles which adhere to the binder-wetted core due to the presence of the binder on the surface of the core.

The polymeric coating mixture may be comprised of any suitable water-insoluble and water-swellable polymers known to those skilled in the art. For example, suitable polymers include: cellulosic polymers, such as methylcellulose (MC), carboxymethylcellulose (CMC), HPC, HPMC, HEC, and the like; vinyl polymers, such as polyvinylpyrrolidone (PVP), polyvinyl alcohol (PVA), and the like; acrylic polymers and copolymers, such as acrylic acid polymer, methacrylic acid copolymers, ethyl acrylate-methyl methacrylate copolymers, and the like; and mixtures thereof. Currently, the preferred polymers include ethylcellulose and HPMC.

HPMC may comprise material of certain viscosity and molecular weight or alternately may comprise mixtures or blends of two or more different forms of HPMC. In one aspect, the mixture may comprise of HPMC having differing molecular weights and solubility characteristics. For example, the mixture may comprise of: a) HPMC having i) a typical weight percent substitution corresponding to about 30% methoxyl and about 10% hydroxypropoxyl groups, and ii) a nominal viscosity of about 2% watery solution at about 20° C. ranging from about 5 to about 100 mPa·s e.g., METHOCEL E5; and b) HPMC having i) a typical weight percent substitution corresponding to about 20% methyoxyl and about 8% hydroxypropoxyl groups, and ii) a nominal viscosity of about 2% watery solution at about 20° C. ranging from about 4,000 to about 100,000 mPa·s (e.g., METHOCEL K15M).

Because the formulations and methods of the present invention may include either a single HPMC or a blend of two or more different forms of HPMC as the coating, for simplicity, the term HPMC as used herein, including the claims, refers to either a single HPMC or a blend of two or more forms of the polymer.

Alternatively, the swellable polymeric coating layer may be comprising of other substances which are functional equivalents to HPMC. For example, polysaccharides, such as gelatin, saccharose, sorbitol, mannanes, and jaluronic acid; polyaminoacids; polyalcohols; polyglycols may also be used.

In addition to the foregoing, the polymeric coating layer may also include other excipients such as lubricants, flow promoting agents, plasticizers, anti-sticking agents, natural and synthetic flavorings and natural and synthetic colorants. Specific examples of additional excipients include polyethylene glycol, polyvinylpyrrolidone, talc, magnesium stearate, glyceryl behenate, stearic acid, and titanium dioxide.

After the powdered polymeric coating layer is applied to the core, the process may be repeated one or more additional times in order to build the thickness of the polymeric coating layer around the core. The number of repetitions is dependent upon the desired predetermined in-vitro dissolution profile and in-vivo performance. A sufficient number of coating cycles are performed so as to produce a core to coating layer weight ratio of between about 20:1 and about 1:5 inclusive, or a thickness in excess of about 10 uM, and up to about 500 uM. In one aspect, a sufficient number of coating cycles are completed so as to produce a core to coating layer weight ratio of between about 5:1 and about 1:3 inclusive, or a thickness ranging from about 50 uM to about 200-400 uM.

The present invention also provides modified release formulations of propranolol that are suitable for oral administration and delivery in the gastro-intestinal tract. A typical formulation includes: (a) a core comprising propranolol hydrochloride, and (b) a polymeric coating layer substantially surrounding the core comprising a mixture of water-insoluble polymer and a water-swellable polymer. As described above, in one aspect, the polymeric coating layer is applied with or without a binder solution or dispersion. The coating cycle may be repeated one or more times to obtain the necessary coating thickness and other criteria to provide the desired in-vitro and in-vivo characteristics.

If desired, the formulations of the present invention may be provided in the form of capsules wherein the core of the present invention is used to fill in a conventional hard or soft-gelatin capsule. Encapsulation within a soft-gelatin capsule is also achievable with conventional techniques. Alternatively, the granules of the invention can be compressed into tablets by using conventional techniques that are well-known in the art.

Additionally, the present invention also provides methods of achieving desired therapeutic benefit from propranolol therapy by administering to the subject the oral dosage forms described herein prepared according to the presently disclosed methods.

### EXAMPLES

The following examples are provided to illustrate the present invention, and should not be construed as limiting thereof. All percentages are in percent by weight of the formulation unless otherwise indicated. Disintegration tests are carried out according to the standard procedures set forth in the United States Pharmacopoeia for testing the disintegration of tablets.

### Example 1

### Granulation

Propranolol HCl (160 mg) and Microcrystalline cellulose powder (101.82 mg) are mixed into a blend with a high shear granulator for 15 minutes. A clear binder solution of ethyl cellulose N100 (8.44 mg) in sufficient amount of Isopropyl alcohol is made. The blend is further granulated with slow addition of the binder solution for 45 min. The granules are dried in a Thelco lab dryer at about 50° C. for about 1 hour. A sufficient amount of water is then added to facilitate extrusion. The resulting mass is extruded through a 1 mm mesh and then spheronized in a spheronizer to create granules. The granules are again dried in a Thelco lab dryer at about 50° C. till the moisture content is less than about 1.0% and solvent content is less than about 0.1% to yield beads with average of 850 um. The beads are then film-coated with a solution of ethylcellulose N100 (14.55 mg) in isopropyl alcohol with triethyl citrate (1.46 mg) as plasticizer in a conventional coating pan. The fill material is then filed into a capsule shell of size “1” with sufficient amount of beads so that the total Propranolol HCl content is 160 mg.

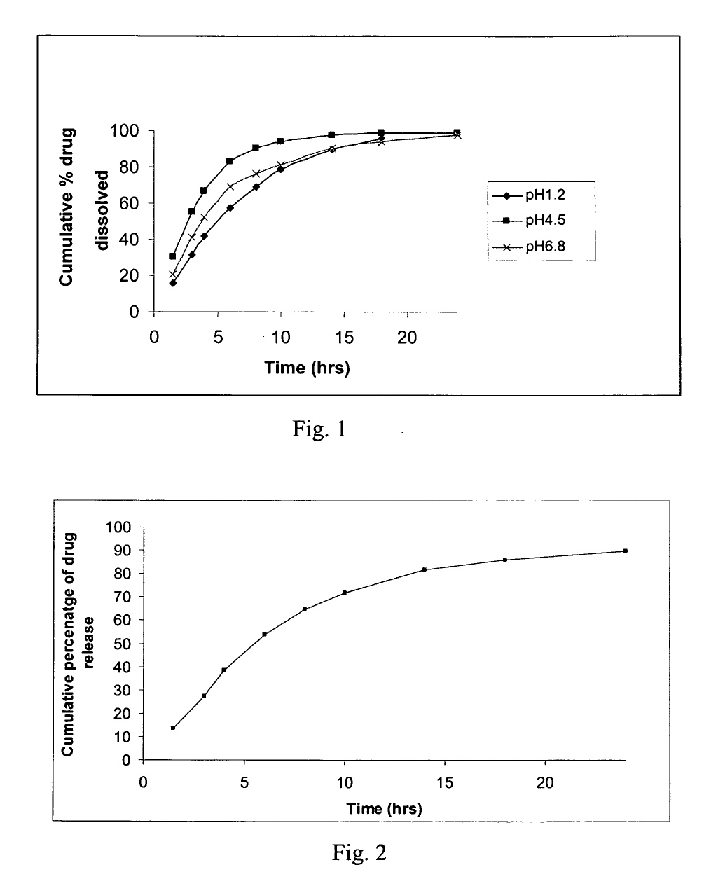

Dissolution is conducted according to the protocol set forth in Example 10. The results thereof are shown in graphical form in FIG. 1.

### Example 2

### Fluid Bed Coating

Propanolol containing cores are prepared as in Example No. 1. The cores containing 160 mg of propranolol hydrochloride are then coated with a solution of Methocel E5 LV premium (3.24 mg) and Ethylcellulose N100 (7.57 mg) in methanol with triethyl citrate (1.08 mg) as plasticizer coating layer using a fluid bed apparatus. A Glatt GPCG 3.1 can be used for this purpose. Fill the capsule size “1” with sufficient amount of beads so that the total Propranolol HCl content is 160 mg.

Dissolution was conducted according to the protocol set forth in Example 10. The results thereof are shown in graphical form in FIG. 2.

### Example 3

Propranolol hydrochloride 120 mg is used per dosage form which may be prepared similar to Example 1, except for the difference in dosage amount and the corresponding differences in the inactive ingredients. Alternatively, the quantity of the beads of Example 1 or 2 may be adjusted proportionately to provide 120 mg of the dose of propranolol hydrochloride.

### Example 4

Propranolol hydrochloride 120 mg is used per dosage form which may be prepared similar to Example 2 except for the difference in dosage amount and the corresponding differences in the inactive ingredients. Alternatively, the quantity of the beads of Example 1 or 2 may be adjusted proportionately to provide 120 mg of the dose of propranolol hydrochloride.

### Example 5

Propranolol hydrochloride 80 mg is used per dosage form which may be prepared similar to Example 1 except for the difference in dosage amount and the corresponding differences in the inactive ingredients. Alternatively, the quantity of the beads of Example 1 or 2 may be adjusted proportionately to provide 80 mg of the dose of propranolol hydrochloride.

### Example 6

Propranolol hydrochloride 60 mg is used per dosage form, which may be prepared similar to Example 1 except for the difference in dosage amount and the corresponding differences in the inactive ingredients. Alternatively, the quantity of the beads of Example 1 or 2 may be adjusted proportionately to provide 60 mg of the dose of propranolol hydrochloride.

### Example 7

Propranolol hydrochloride 200 mg may be prepared similar to Example 1 except for the difference in dosage amount and the corresponding differences in the inactive ingredients. Alternatively, the quantity of the beads of Example 1 or 2 may be adjusted proportionately to provide 200 mg of the dose of propranolol hydrochloride.

### Example 8

Propranolol hydrochloride 240 mg may be prepared similar to Example 1 except for the difference in dosage amount and the corresponding differences in the inactive ingredients. Alternatively, the quantity of the beads of Example 1 or 2 may be adjusted proportionately to provide 240 mg of the dose of propranolol hydrochloride.

### Example 9

Propranolol hydrochloride 300 mg may be prepared similar to Example 1 except for the difference in dosage amount and the corresponding differences in the inactive ingredients. Alternatively, the quantity of the beads of Example 1 or 2 may be adjusted proportionately to provide 300 mg of the dose of propranolol hydrochloride.

### Example 10

Propranolol hydrochloride 1 60 mg may be prepared in an alternative method to Example 1. The dried granules are, instead of being subjected to extrusion/spheronization, lubricated with any of the generally known lubricants in the art, and compressed into minitablets of desired shape and size. These minitablets are then coated as in Example 1, which coated minitablets are then used to fill in a capsule to provide the desired dissolution profile as exemplified in Tables 2-4.

### Example 11

To validate the robustness of the present invention in terms of coating composition, coating methodology and commercial feasibility, in-vitro dissolution tests in so-called “discriminating media” under different pH values are conducted. The details of these experiments are shown below in Table 1 for dosage form presented in Example 1.

TABLE 1

| | BUFFER | | SPEED | | | PATH- | WAVE- |
|---|---|---|---|---|---|---|---|
| NO. | CONCENTRATION | VOLUME | (RPM) | TEMPERATURE | APPARATUS | LENGTH | LENGTH |
| 1 | pH 1.2 SGF w/o pepsin | 900 | 100 | 37 +/− 0.5 C. | 1, BASKETS | 0.1 | 320 |
| 2 | pH 4.5 ACETATE | 900 | 100 | 37 +/− 0.5 C. | 1, BASKETS | 0.1 | 320 |
| 3 | pH 6.8 Sod Phosphate | 900 | 100 | 37 +/− 0.5 C. | 1, BASKETS | 0.1 | 320 |
| | dibasic + Citric Acid | | | | | | |
| 4 | pH 7.5 PHOSPHATE | 900 | 100 | 37 +/− 0.5 C. | 1, BASKETS | 0.1 | 320 |

Sampling points:- 1.5, 3, 4, 6, 8, 10, 14, 18, 24 hrs

The dissolution profiles indicated that the modified release propranolol formulations of the present invention are equivalent to the branded Inderal LA formulations. This equivalency is robust, and is reproducible in discriminating media among various pH values. This result is quite unexpected and surprising yet highly desirable.

The present invention has provided modified release propranolol formulations that are comparable to the branded INDERAL LA® product under simulated fed conditions, assuring greater confidence for success in actual clinical studies.

These sample products were subjected to in-vitro dissolution testing under various conditions. The specific dissolution results are outlined in the Tables 2-4 below. The data are displayed in graphical form in FIG. 1.

TABLE 2
In-vitro Dissolution profile of Propranolol
HCl Long Acting/160 mg Capsules pH 1.2

| Time (hrs) | pH 1.2 Cumulative % released |
|---|---|
| 1.5 | 21.9 |
| 3 | 44.34 |
| 4 | 56.58 |
| 6 | 68.44 |
| 8 | 85.8 |
| 10 | 94.08 |
| 14 | 100.09 |
| 18 | 102.49 |
| 24 | 103.42 |

TABLE 3
In-vitro Dissolution profile of Propranolol
HCl Long Acting/160 mg Capsules pH 4.5

| Time (hrs) | pH 4.5 Cumulative % released |
|---|---|
| 1.5 | 43.26 |
| 3 | 74.27 |
| 4 | 85.47 |
| 6 | 95.66 |
| 8 | 99.36 |
| 10 | 100.92 |
| 14 | 101.95 |
| 18 | 102.38 |
| 24 | 103.52 |

TABLE 4
In-vitro Dissolution profile of Propranolol
HCl Long Acting/160 mg Capsules pH 6.8

| Time (hrs) | pH 6.8 Cumulative % released |
|---|---|
| 1.5 | 19.84 |
| 3 | 39.71 |
| 4 | 49.08 |
| 6 | 58.07 |
| 8 | 73.15 |
| 10 | 80.53 |
| 14 | 87.33 |
| 18 | 91.21 |
| 24 | 94.61 |

### Example 12

Dissolution testing was conducted according to the official methodology in United States Pharmacoepia 27, monograph titled “Propranolol Hydrochloride Extended Release Capsules,” which is incorporated by reference. The conditions described therein are generally known as USP conditions. Typically, this comprises conducting the dissolution at a pH of 1.2 by using HCl and sodium chloride for the first 90 minutes, followed by a pH of 6.8 for the rest of the experiment. Generally, the pH 6.8 is achieved by using sodium phosphate and citric acid. Briefly, for each test, either one capsule of branded product, INDERAL® LA or one capsule of the present invention (designated as CPI) with 160 mg of equivalent active cores is used. 8 mL samples are withdrawn at predetermined times using an automated sampler. The Propranolol HCl concentration in each sample was determined using an UV-V is spectrophotometer at wavelength of 320 for all dissolution media. The percentage of Propranolol HCl released over time is calculated and plotted as an average of 6 runs using calibration curves consistent with Beer's law.

Further experimental details are provided as following in Table 5.

TABLE 5

| | BUFFER | | SPEED | | | PATH- | WAVE- |
|---|---|---|---|---|---|---|---|
| NO. | CONCENTRATION | VOLUME | (RPM) | TEMPERATURE | APPARATUS | LENGTH | LENGTH |
| 1 | USP CONDITIONS | 900 | 100 | 37 ± 0.5 C. | 1, BASKETS | 0.1 | 320 |
| | (pH 1.2 for 2 hrs | | | | | | |
| | followed by pH 6.8) | | | | | | |

Sampling points:- 1.5, 3, 4, 6, 8, 10, 14, 18, 24 hrs

The resulting dissolution data are presented in Table 6 and in graphical form as FIG. 2. The data indicate that the propranolol modified dosage form as formulated and prepared according to the presented invention has met the Official USP dissolution requirements.

TABLE 6
In-vitro Dissolution profile of Propranolol HCl Long
Acting/160 mg Capsules (USP media (1.5 hrs in pH 1.2
followed by pH 6.8 for the rest of the time)

| Time (hrs) | Cumulative % Release Formulation Example 1 |
|---|---|
| 1.5 | 21.04 |
| 3 | 37.71 |
| 4 | 44.26 |
| 6 | 61.74 |
| 8 | 72.35 |
| 10 | 78.18 |
| 14 | 84.47 |
| 18 | 88.61 |
| 24 | 93.04 |

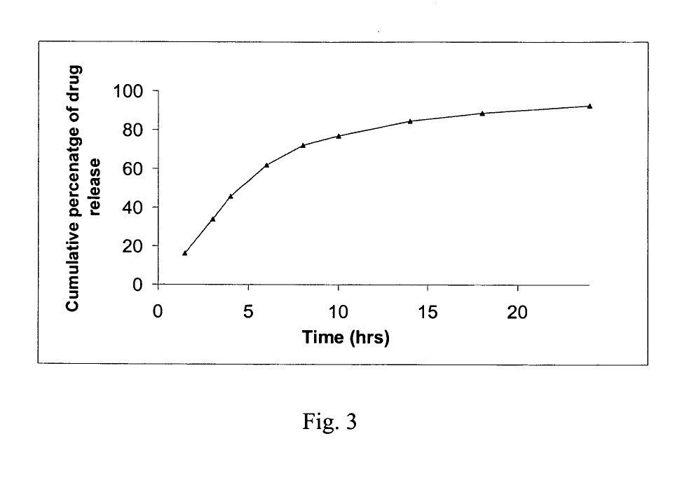

Another sample of product prepared according to Example 1 was subjected to in-vitro dissolution testing under various conditions. The specific dissolution results are outlined in Table 7 below and in graphical form as FIG. 3.

TABLE 7
In-vitro Dissolution profile of Propranolol HCl Long
Acting/160 mg Capsules (USP media (1.5 hrs in pH 1.2
followed by pH 6.8 for the rest of the time)

| Time (hrs) | Cumulative % Release Formulation of Example 1 |
|---|---|
| 1.5 | 12.71 |
| 3 | 25.72 |
| 4 | 35.49 |
| 6 | 52.04 |
| 8 | 63.77 |
| 10 | 73.08 |
| 14 | 81.95 |
| 18 | 86.4 |
| 24 | 90.68 |

The foregoing is illustrative of the present invention and is not to be construed as limiting thereof. The invention is defined by the following claims, with equivalents of the claims to be included therein.

What is claimed is:

1. A modified release propranolol oral dosage formulation comprising:
a) a therapeutically effective amount of propranolol, ranging from about 60 mg to about 300 mg per dosage unit, formulated into one or more cores comprising said propranolol and one or pharmaceutically acceptable excipients;
b) a release-modifying coat that substantially or completely overlaps said core, wherein said coat comprises a mixture of a water-impermeable polymer and a water-swellable polymer; to provide a dissolution profile selected from the group consisting of:
i) about 10-30% of the drug is released by 90 minutes; about 30% to about 60% of the drug is released by 4 hrs; and about 50% to about 80% of the drug is released by 8 hrs; when dissolution test is performed using pH 6.8 phosphate buffer and simulated intestinal fluid without pancreatin, using Type 1 dissolution apparatus being operated at about 37° C., using a volume of about 900 ml of the dissolution medium being stirred at a speed of 100 rpm;
ii) about 20% to about 45% of the drug is released by 90 minutes; about 40% to about 75% of the drug is released by 4 hrs; about 60% to about 90% of the drug is released by 8 hrs, when dissolution test is performed using pH 1.2 simulated gastric fluid without pepsin, using Type 1 dissolution apparatus being operated at about 37° C., using a volume of about 900 ml of the dissolution medium being stirred at a speed of 100 rpm;
iii) about 20 to about 50% of the drug is released by 90 minutes; about 60% to about 90% of the drug is released by 4 hrs; about 70% to about 100% of the drug is released by 8 hrs when dissolution test is performed using pH 4.5 phosphate buffer, using Type 1 dissolution apparatus being operated at about 37° C., using a volume of about 900 ml of the dissolution medium being stirred at a speed of 100 rpm; and
iv) about 10-25% of the drug is released by 90 minutes; about 25-55% of the drug is released by 4 hrs; about 40-70% of the drug is released by 6 hrs; and about 60% to about 80% of the drug is released by 8 hrs, under USP dissolution conditions with a pH of 1.2 for 2 hours followed by a pH of 6.8 for the rest of the time, using Type 1 dissolution apparatus being operated at about 37° C., using a volume of about 900 ml of the dissolution medium being stirred at a speed of 100 rpm.

2. The formulation of claim 1, wherein the at least one pharmaceutically acceptable excipient is a member selected from the group consisting of: microcrystalline cellulose, dibasic calcium phosphate dihydrate, starch, sodium starch glycolate, crospovidone, croscarmellose sodium, magnesium stearate, lactose, maleic acid, colloidal silicon dioxide, talc, glyceryl behenate, and mixtures thereof.

3. The formulation of claim 3, wherein the at least one pharmaceutically acceptable excipient is microcrystalline cellulose.

4. The formulation of claim 1, wherein the cores comprise a mixture of at least one water-impermeable polymer and at least one water-swellable polymer.

5. The formulation of claim 4, wherein the cores do not comprise an inert bead.

6. The formulation of claim 1, wherein the formulation comprises propranolol comprising cores with a particle size ranging from about 800 uM to about 1200 uM.

7. The formulation of claim 1, wherein the water insoluble polymer is a member selected from the group consisting of: ethylcellulose, propylcellulose, isopropylcellulose, or a mixture thereof.

8. The formulation of claim 5, wherein the water swellable polymer is a member selected from the group consisting of: methylcellulose (MC), carboxymethylcellulose (CMC), hydroxypropylcellulose (HPC), hydroxypropylmethylcellulose (HPMC), hydroxyethylcellulose (HEC); polyvinylpyrrolidone (PVP), polyvinyl alcohol (PVA); and acrylic acid polymer, methacrylic acid copolymers, ethyl acrylate-methyl methacrylate copolymers, and mixtures thereof.

9. The formulation of claim 8, wherein the water swellable polymer is methylcellulose.

10. The formulation of claim 5, wherein the coating comprises a mixture of a water insoluble polymer and a water swellable polymer, and the water insoluble polymer is ethylcellulose and the water swellable polymer is hydoxypropylmethylcellulose.

11. A method of making a modified release propranolol formulation as recited in claim 1, comprising:
a) preparing a mixture of propranolol hydrochloride and one or more pharmaceutically acceptable excipients to form a propranolol-excipient mixture;
b) forming the propranolol-excipient mixture into cores; and
c) coating said propranolol-excipient cores with one or more polymers.

12. The method of claim 11, wherein the mixture of propranolol and one or more pharmaceutically acceptable excipients is prepared by granulating the propranolol-excipient mixture in the presence of a water-insoluble polymer to produce propranolol granulates.

13. The method of claim 12, wherein forming the propranolol-excipient mixture into cores further comprises either spheronizing and extruding the propranolol granulates to produce propranolol cores or compressing into cores in the form of minitablets.

14. The method of claim 13, wherein the cores are dried and sieved.

15. The method of claim 11, wherein the coating of the cores comprises:
a) preparing a dispersion of a water-insoluble polymer and a water-swellable polymer to produce a coating polymer dispersion;
b) coating said propranolol cores with said coating polymer dispersion to obtain coated propranolol cores; and
c) provide modified release propranolol capsules by filling empty capsules with coated propranolol cores.

16. A method of administering propranolol therapy to a subject in need thereof, comprising:
administering a therapeutically effective amount of propranolol rom polymer coated propranolol containing cores which provide a peal propranolol blood plasma concentration at about 6 hours after administration.

17. The method of claim 16, the propranolol therapy is used to treat a condition selected from the group consisting of: hypertension, angina, migraine, hypertrophic subaortic stenosis, and combinations thereof.

18. The method of claim 17, wherein the condition is either hypertension or angina, or both.

19. A modified release propranolol oral dosage formulation comprising:
a) a therapeutically effective amount of propranolol, ranging from about 60 mg to about 300 mg per dosage unit, formulated into one or more cores comprising said propranolol and one or pharmaceutically acceptable excipients;
b) a release-modifying coat that substantially or completely overlaps said core, wherein said coat comprises a mixture of a water-impermeable polymer and a water-swellable polymer;
wherein, the dosage form provides a dissolution profile selected from the group consisting of:
i) about 10-30% of the drug is released by 90 minutes; about 30% to about 60% of the drug is released by 4 hrs; and about 50% to about 80% of the drug is released by 8 hrs; when dissolution test is performed using pH 6.8 phosphate buffer and simulated intestinal fluid without pancreatin, using Type 1 dissolution apparatus being operated at about 37° C., using a volume of about 900 ml of the dissolution medium being stirred at a speed of 100 rpm;
ii) about 20% to about 45% of the drug is released by 90 minutes; about 40% to about 75% of the drug is released by 4 hrs; about 60% to about 90% of the drug is released by 8hrs, when dissolution test is performed using pH 1.2 simulated gastric fluid without pepsin, using Type 1 dissolution apparatus being operated at about 37° C., using a volume of about 900 ml of the dissolution medium being stirred at a speed of 100 rpm;
iii) about 20 to about 50% of the drug is released by 90 minutes; about 60% to about 90% of the drug is released by 4 hrs; about 70% to about 100% of the drug is released by 8 hrs when dissolution test is performed using pH 4.5 phosphate buffer, using Type 1 dissolution apparatus being operated at about 37° C., using a volume of about 900 ml of the dissolution medium being stirred at a speed of 100 rpm; and
iv) about 10-25% of the drug is released by 90 minutes; about 25-55% of the drug is released by 4 hrs; about 40-70% of the drug is released by 6 hrs; and about 60% to about 80% of the drug is released by 8 hrs, under USP dissolution conditions with a pH of 1.2 for 2 hours followed by a pH of 6.8 for the rest of the time, using Type 1 dissolution apparatus being operated at about 37° C., using a volume of about 900 ml of the dissolution medium being stirred at a speed of 100 rpm; and,
wherein said dosage form provides a peak blood plasma concentration at about 6 hrs after administration to a subject.

20. A method of making a modified propranolol dosage formulation as recited in claim 19 comprising:
a) preparing a mixture of propranolol hydrochloride and one or more pharmaceutically acceptable excipients to form a propranolol-excipient mixture;
b) granulating the propranolol-excipient mixture in the presence of a water-insoluble polymer to produce propranolol granulates;
c) spheronizing and extruding the propranolol granulates to produce propranolol cores, and optionally drying and sieving said cores;
d) preparing a dispersion of a water-insoluble polymer and a water-swellable polymer to produce a coating polymer dispersion;
e) coat said propranolol cores with said coating polymer dispersion to obtain coated propranolol cores; and
f) providing modified release propranolol capsules by filling empty capsules with coated propranolol cores.
